(19) United States
(12) Patent Application Publication
(10) Pub. No.: US 2020/0352258 A1
(43) Pub. Date: Nov. 12, 2020

(54) ATHLETIC TRAINING APPAREL

(71) Applicants: Metacaum M Evans, Leavenworth, KS (US); Jueon Park, Leavenworth, KS (US)

(72) Inventors: Metacaum M Evans, Leavenworth, KS (US); Jueon Park, Leavenworth, KS (US)

(21) Appl. No.: 16/407,341

(22) Filed: May 9, 2019

(51) Int. Cl. A41D 1/08; A41D 1/00; A41D 13/00

(52) U.S. Cl. CPC A41D 1/08; A41D 1/002; A41D 2600/10; A61B 2503/10; A41D 13/0015

(57) ABSTRACT

An apparel for athletic training is presented. The apparel is useful for helping to reduce fungal and bacterial infections resulting from most athletic apparel. The apparel may also be useful by enhancing low extremity circulation thus helping to preventing varicose veins while protecting the knees, tail bone and hips from trauma. The athletic training apparel 100 is preferably made of antimicrobial fabric. The apparel may have a knee pad to protect each knee, hip pads to protect the hips and a tail bone pad for protecting the tail bone of a trainee. Further a first and second footing for providing security and comfort while training may have a carbon fiber covering for the bottom of each of the footings.

### FIELD OF THE INVENTION

This invention relates to athletic training. More particularly, it relates to apparel for athletic training.

### BACKGROUND

Athletic training has been recognized by the American Medical Association (AMA) as an allied health care profession since June 1991.

Athletic training is practiced by athletic trainers, health care professionals who collaborate with physicians to optimize activity and participation of patients and clients. Athletic training encompasses the prevention, diagnosis, and intervention of emergency, acute and chronic medical conditions involving impairment, functional limitations and disabilities.

There are five domains of athletic training listed in the 7th edition (2015) of the Athletic Training Practice Analysis:
Injury and Illness Prevention and Wellness Promotion
Examination, Assessment and Diagnosis
Immediate and Emergency Care
Therapeutic Intervention
Healthcare Administration and Professional Responsibility

An athletic trainer functions as an integral member of the health care team in clinics, secondary schools, colleges and universities, professional sports programs, and other athletic health care settings.

When addressing injury and illness prevention and promoting wellness, there has been little movement to protect the athlete's joints and muscles in the clothing designed for training. Therefore, there is a need for an athletic training tights that can help protect the body of the training athlete.

### BRIEF DESCRIPTION OF THE DRAWINGS

FIG. 1A is an illustrated view of an exemplary athletic training apparel.

FIG. 1B is an illustrated detailed view of the footings of the athletic training apparel of FIG. 1A.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

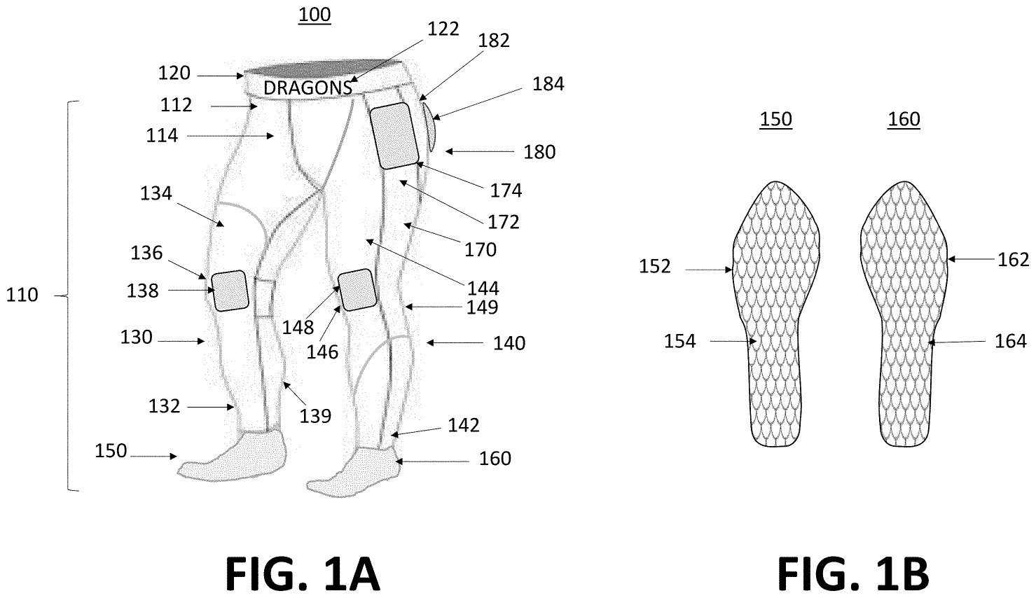

Referring to FIG. 1A and FIG. 1B, an exemplary athletic training apparel 100 for protection during athletic training is presented. The apparel 100 is useful for protecting the joint areas of a trainee's body while participating in athletic training.

The apparel 100 is also useful for helping to reduce fungal and bacterial infections resulting from most athletic apparel. The apparel may also be useful by enhancing low extremity circulation thus helping to preventing varicose veins while protecting the knees, tail bone and hips from trauma. The athletic training apparel 100 is preferably made of antimicrobial fabric. Antimicrobial fabrics can be made of a variety of textiles, including but not limited to polyester, polyester-vinyl composites, vinyl, acrylics, etc.

The apparel 100 is preferably pants shape, but may be other shapes, such as shorts, pajamas, etc. The apparel 100 is preferably a tight fit, such as tights, but may be loose fit as desired.

The apparel 100 has a body 110 and a waist band 120. The body 110 is the entire shape of the apparel 100. The body 110 is shaped to be consistent with a body of a person. The body 110 has a first leg 130, a second leg 140, a first footing 150, a second footing 160, two side areas 170 and a rear portion 180.

The waist band 120 is coupled to an upper portion 112 of the body 110 of the apparel 100. The first leg 130 has a distal end 132, a front side 134, a knee portion 136 and a back portion 137. The first leg 130 may have a knee pad 138. The knee pad 138 is useful in protecting a knee area of the trainee when the trainee's knee is against the ground or hit upon an item. The knee pad 138 of the first leg 130 is preferably a soft padding such as foam, but may be any other protective material such as cotton, denim, etc. The waist band 120 may have a brand name or other logo 122.

The second leg 140 has a distal end 142, a front side 144, a knee portion 146 and a back portion 147. The second leg 140 may have a knee pad 148. The knee pad 148 is useful in protecting a knee area of the trainee when the trainee's knee is against the ground or hit upon an item. The knee pad 148 of the second leg 140 is preferably a soft padding such as foam, but may be any other protective material such as cotton, denim, etc.

The first footing 150 is coupled to the distal end 132 of the first leg 130 of the apparel 100. The first footing 150 is useful in providing a stable and comfortable surface while the trainee is performing athletic training exercises.

The first footing 150 in FIG. 1B, show the first footing 150 having a bottom side 152 and a bottom covering 154. The bottom covering 154 is coupled to the bottom side 152 of the first footing 150. The coupling of the bottom covering is preferably glue, but may be Velcro, stitching, etc. The bottom cover 154 of the first footing 150 is preferably carbon rubber, which is hard, or blown rubber, a softer type, etc.

The second footing 160 is coupled to the distal end 142 of the second leg 140 of the apparel 100. The second footing 160 is useful in providing a stable and comfortable surface while the trainee is performing athletic training exercises.

The second footing 160 in FIG. 1B, show the second footing 160 having a bottom side 162 and a bottom covering 164. The bottom covering 164 is coupled to the bottom side 162 of the second footing 160. The coupling of the bottom covering is preferably glue, but may be Velcro, stitching, etc. The bottom cover 164 of the second footing 160 is preferably carbon rubber, which is hard, or blown rubber, a softer type, etc. but may be other materials as desired.

Each of the two side areas 170 of the body 110 of the apparel 100 is located between the rear portion 180 of the body of the apparel 100 and a front side 114 of the apparel 100. Each of the two sides 170 has a hip portion 172 where the hip portion 172 covers the outside of each of the hips of the trainee.

The apparel 100 may further have two hip pads 174. The two hip pads 174 protect the two hips of the trainee while the trainee is in athletic training. Each of the two hip pads 174 are coupled to each of the hip portions 172 of each of the two sides 170 of the body 110 of the apparel 100.

The rear portion 180 of the body 110 of the apparel 100 covers the rear end of the trainee while in athletic training. The rear portion 180 of the body 110 of the apparel 100 has a tail bone area 182. The tail bone area 182 covers and protects a tail one of the trainee. To further protect the tail bone of the trainee, the tail bone area 182 may have a tail bone pad 182. The tail bone pad 182 further protects the tail bone of the trainee while in athletic training as the tail bone when injured is painful and can cause physical diminishing. The tail bone pad 184 of the rear portion 180 is preferably a soft padding such as foam, but may be any other protective material such as cotton, denim, etc.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An apparel for preventing injuries during athletic training, the apparel comprising:
a body, the body for protecting a trainee in athletic training, the body having a first leg, a second leg, a rear side and two side areas;
a waist band, the waist band coupled to an upper portion of the body;
the first leg having a distal end and a knee portion, wherein the knee portion being coupled to a knee pad and wherein the distal end being coupled to a first footing;
the second leg having a distal end and a knee portion, wherein the knee portion being coupled to a knee pad and wherein the distal end being coupled to a second footing;
two sides areas, the two sides being coupled to the rear side and the front side of the apparel, wherein the two sides having a hip portion;
a hip pad, the hip pad for protecting the hip of the trainee, wherein the hip pad being coupled to the hip portion of each of the two side areas; and
a rear portion, the rear portion having a tail bone area and wherein the tail bone area being coupled to a tail bone pad to protect the tail bone from trauma.

2. The apparel of claim 1, wherein the apparel being made of an antimicrobial fabric.

3. The apparel of claim 2, wherein the antimicrobial fabric being a polyester material.

4. The apparel of claim 1, wherein the first footing having a bottom and wherein the bottom having a covering.

5. The apparel of claim 4, wherein the covering being made of a carbon rubber material.

6. The apparel of claim 4, wherein the covering is coupled to the bottom by a glue material.

7. The apparel of claim 1, wherein the knee pad being made of a foam material.

8. The apparel of claim 1, wherein the hip pad being made of a foam material.

9. The apparel of claim 1, wherein the tail bone pad being made of a foam material.
